(19) United States
(12) Patent Application Publication
(10) Pub. No.: US 2004/0176838 A1
(43) Pub. Date: Sep. 9, 2004

(54) Medical device

(76) Inventors: Andreas Mucha, Bannewitz (DE); Manfred Gulcher, Raesfeld (DE); Martina Nissl, Garstedt (DE)

Correspondence Address: HENRY M FEIEREISEN, LLC, 350 FIFTH AVENUE, SUITE 4714, NEW YORK, NY, 10118, US

(21) Appl. No.: 10/478,342

(22) PCT Filed: May 21, 2002
(86) PCT No.: PCT/DE02/01856

Related U.S. Application Data
(60) Provisional application No. 60/292,434, filed on May 21, 2001.

(51) Int. Cl. A61F 2/06

(52) U.S. Cl. 623/1.45; 623/1.46

(57) ABSTRACT

The invention relates to a medical device for placement in the body, in particular in a body vessel of a patient. The device includes a substrate (1), which is suitable for long-term placement, whose biocompatible properties have been modified by bombarding its surface (2) with foreign ions (3). The foreign ions (3) are thus embedded in the substrate (1) and form a boundary layer (5) that inhibits the diffusion of substrate ions (4) between the surface (2) and the interior (6) of the substrate (1).

The invention relates to a medical device for placement in a body vessel of a patient according to the features of the preamble of claim 1.

In particular, the invention relates to vascular or cardiovascular stents.

Vascular diseases rank as the main causes for disabilities and death in industrial countries. In the United States, more than half of all causes of death are cardiovascular diseases. The most known form of vascular diseases is arteriosclerosis which causes the insufficient blood supply of organs. Heart attacks, stroke and kidney failure may be the outcome. Arteriosclerosis may be the result of vascular injury in which the vascular smooth muscle cells of an arterial wall are subjected to a hyperproliferation and invaded by inner vessel mucous which spreads there. As a consequence, they are completely blocked in the case of a local blood clotting, resulting in a dying of the tissue supplied by this artery. If a coronary artery is involved here, the obstruction may lead to a heart attack and death.

Blockage of the coronary artery may be treated through a bypass of the coronary artery and/or angioplasty. Although both procedures show promise initially; they are practically useless in the event restenosis is experienced after such a treatment. It is assumed that restenosis also involves a hyperproliferation of vascular smooth muscle cells. A third of patients treated by angioplasty encounter restenosis and blockage within six months after treatment, and it has been shown that the rate of restenosis is significantly higher in some patient groups (diabetics, smokers).

While the outcome of the restenosis is the same (loss of intraluminal space), it is assumed that the mechanism in case of percutaneous transluminal coronary angioplasty (PTCA) and stenting is different. A main cause for restenosis after a PTCA is a stenotic blockage through elastic reconfiguration of the vessel wall, while a loss in lumen as a consequence of non-growth of tissue cells in the intraluminal space (intimal hyperplasia) is less dominant. In case of stented vessels, the restenosis is dominant because of the intimal hyperplasia.

After an injury caused by a balloon during angioplasty or stenting, the vessel is bare—cleared from the endothelial layer. This results in an increased leucocytic proliferation, separated in neutrophilia and mononucleocytes in order to eliminate the cellular fragments (scavenge). The activation of these cells results in a liberation of various promoting factors (cytokines) which have proven to trigger a proliferation of smooth muscle cells. A lowering of these factors stops the proliferation. During persistent injury, a lowering is not encountered, leading to vascular hyperplasia and restenosis. The extent and type of the inflammation correlate therefore directly with the extent and degree of the restenosis through cellular hyperplasia (smooth muscle cell proliferation).

In vessels that undergo solely PTCA, neutrophilia only has been discerned but no macrophages. This is a clear indication of no foreign-body reaction.

Other studies have shown that PTCA does not cause any chronic injury in comparison to stenting. However, numerous foreign-body reactions have been encountered in stented vessels. High concentration of macrophages as well as tissue granulations has been ascertained. This is an indication that a foreign-body reaction has taken place in addition to the wound healing. Cause may be the stent material.

One study established a close correlation between the in-stent restenosis and contact-based allergies against metals. In view of this discovery, the material selection for stents and its negative property to cause a localized inflammatory reaction as well as also a system-inherent allergic reaction becomes very important. Stents, for example, which are coated with a biocompatible layer cause more often inflammatory reactions and hyperplasia than uncoated stents.

Most currently used stent products are made of special steel or nitinol. Extensive in-vivo and in-vitro studies confirm a good biocompatibility of these metal alloys. On the other hand, inflammatory and allergic reactions in connection with these metal implants have been amply documented. Collected data about patients with a metallic hip implant have shown a direct correlation between elevated amounts of chromium ions and nickel ions, on the one hand, and a reduction of white blood cells, on the other hand. The entire immune system has been weakened. Even three years after an arthroplasty, significantly elevated concentrations of metallic ions had been found in patients in blood serum as well as also in the urine.

Nitinol contains a high concentration of nickel. Special steel contains nickel, chromium and molybdenum. Acute toxicity and cytotoxicity of ions of these metals was shown. Several studies that have been undertaken to evaluate the effect of escaping metallic ions from nitinol and special steel confirm the toxic effect of high ionic concentrations on various in-vitro cell cultures, including fibroblasts, epithelial cells and muscle cells. Tests have shown that although small concentrations of escaping Ni-ions do not cause any activation of ICAM (Improved Chemical Agent Monitor/intercellular adhesion molecules) on the surface of the endothelial cells, these levels are however sufficient to cause the liberation of IL-6 from monocytes, leading indirectly to an activation of ICAM. This fact is very important because activated endothelial cells are responsible for an increase of inflamed blood cells. As a consequence, an increased amount of inflamed cells remains in the area of the stent and promotes further neointimal proliferation.

All these studies that have been carried out with varying toxic concentration amounts for Ni-ions and Cr-ions lead to the same conclusion that the cause for these ions is a type of corrosion process which causes the alloy to wash out. Therefore, the long-term resistance to corrosion of the stent material becomes an important aspect for the decrease of in-stent restenosis.

The corrosion resistance of metallic implants is based on their passivation through a thin oxide layer. Several surface passivation techniques have been considered for the stent use. The common conclusion of all these studies is that a better surface passivation significantly impairs/reduces the escape of metallic ions.

A further possibility to control the growth of cells into the vessel lumen involves making the stent radioactive. Hereby, radioisotopes are used with high decay energy to limit the effect of radiation on the immediate environment of the stent. However, there is the problem that while the restenosis can be almost entirely suppressed in the area of the radioactive stent, a strong tissue growth is experienced at the ends of the stent. The negative effect of the radioactive stent may be caused by an insufficient dose of irradiation on the stent ends as a consequence of the short range of the p-radiation. This problem can be resolved only by a higher activity on the stent ends. Furthermore, a typically used ball-type implant catheter is longer on both ends by about 2 to 3 mm than the stent to prevent a loss when being inserted into the stenosis. This may lead to injury of the arterial wall at a fairly great distance from the stent.

Further approaches to prevent restenosis include the introduction of radioactivity before or after the balloon dilatation. It has been shown that the irradiation of the vessel wall is able to significantly reduce the cell growth after a balloon dilatation. A drawback hereby is the introduction of a strong radioactive source into the body of the patient in order to expose the vessel wall to a respectively high radiation dose within a short time. The surgical team and patients are exposed in this procedure to a higher level of radiation compared to the use of a radioactive stent of lesser activity.

Radioactive stents can be made by the process of ion implantation in which a particular ion source is used to shoot ions into the stent material. The radioactivity is localized below the surface of the stent material.

It is also known to passivate stents through application of layers. Certain metals, such as iron, chromium, nickel, and possibly their alloys react very slowly in the presence of a surface film which acts as protection against corrosion. Special steel, for example, is refined by the thin protective chromium layer. This type of the passive film depends predominantly on the metal and the conditions by which the film has been produced.

Practice has shown that coatings applied onto an implanted medical device, such as, e.g., a stent placed into the body of the patient, not always have the desired adhesion to the substrate of the stent so that the surface of the stent changes and metal alloys may wash out, when the substrate of the stent is exposed, resulting in the afore-described disadvantageous consequences.

Application of passivating layers in stents poses especially a problem because the stents are subjected to a significant mechanical stress during placement in the body lumen. In view of varying material properties of the coating and the substrate, cracks in the coating cannot be excluded or the coating may even peel off.

It is therefore the object of the invention to provide a medical device for placement in the body of a patient, which is improved as far as passivation of the surface of its substrate is concerned to thereby exhibit better biocompatible/hemocompatible properties.

This object is attained by the invention through provision of a medical device according to the features of claim 1.

The essence of the invention is the embedding of foreign ions in the substrate of the medical device to form a diffusion-inhibiting boundary layer between the surface and the interior of the substrate.

The ion implantation is a vacuum process in which the solid surfaces are bombarded by the charged elementary particles (ions) at high energy. The ions penetrate the surface-proximate regions of the substrate in the form of metal ions, noble gas ions or, as here, in the form of reactive ions. Unlike ion beam irradiation, the plasma immersion ion implantation, the ion implantation does not involve the “application” of an additional layer onto the substrate, but atomic components are “embedded” below the substrate surface. An advantage is hereby the superior dimensional stability of the refined surfaces. In particular, there is no problem with respect to adhesiveness as known from other coating processes.

Particular advantages which distinguish the ion implantation from other thin-layer technologies include further the retention of the finish, the low process temperature as well as the variety of the substrate materials (metals, plastics, ceramics) and the type of foreign ions. Alloys can be made even at high reproducibility outside the thermal equilibrium. The finishing process may, e.g., involve a smoothening of the surface by means of electrical or mechanical polishing agents.

The ion implantation further involves an environmentally compatible process which is harmless as far as especially irradiation problems are concerned as can be experienced with radioactive stents. A further advantage involves the extremely slight depth of penetration of the incorporated foreign ions into the substrate so that the mechanical properties of the substrate remain largely unchanged.

In accordance with the claimed medical device, the entire surface of the substrate is bombarded by foreign ions at relatively high energy under vacuum and penetrated. In other words, the foreign ions replace either the free spaces in the lattice structure of the substrate or dislodge other atoms from the surface-proximate spaces of the substrate and replace these spaces. An essential enrichment of the foreign ion concentration does not occur because only free or freed lattice spaces are replaced by foreign ions.

Depending on the level of the energy dose, substrate ions, in particular nickel ions, are more or less deeply pushed back in direction toward the interior of the substrate. The diffusion-inhibiting boundary layer can therefore be formed at a distance to the substrate surface also further within the substrate. Thus, the diffusion-inhibiting boundary layer does not necessarily have to be located directly at or below the surface. Preferably, a high energy dose is selected in order to shift substrate ions, in particular the element nickel, towards deep enough layers.

Through variation of the implantation energy, it is further possible to enrich, in addition to the boundary layer located inside the substrate, the outer surface of the substrate with foreign ions in such a manner that the concentration of the foreign ions at the surface or in immediate surface-proximate regions is greater than 90%. The concentration may even be greater than 95%. Through selection of suitable foreign ions, the biocompatibility of the substrate is decisively improved. Especially advantageous is hereby the combination of diffusion-inhibiting boundary layer in the deeper situated interior of the substrate with a biocompatible layer provided directly beneath the surface.

Tests with carbon ions as foreign ions, I.e. a carbonation of a substrate, in particular a stent, have shown that mainly heavy metals, in particular nickel, are dislodged and replaced by the body-own and thus highly biocompatible carbon.

Depending on the substrate, the depth of penetration of the carbon ranges between few nanometers and up to about 20 microns, thus constituting a very small depth of penetration compared to the material thickness of a common stent so that the substrate, as viewed in its entirety, retains its mechanical properties. The device according to the invention can therefore also include a substrate with very specific physical properties, like for example nitinol.

Foreign ions may be ions of a single element, like for example carbon, oxygen, nitrogen or tantalum, whereby the latter improves the X-ray visibility at the same time. Ions of the mentioned elements have the positive effect to dislodge metal ions, in particular heavy metal ions.

Advantageous embodiments of the inventive concept are set forth in the sub-claims.

According to claim 2, the foreign ions may also be a mixture of ions of several elements, for example, of carbon and oxygen. Relevant hereby is a particular impact of this mixture of foreign ions upon the composition of the diffusion-inhibiting boundary layer. When initially introducing foreign ions of a first element through ion implantation into the medical device and subsequently introducing foreign ions of a second element, there would be the consequence that the foreign ions of the second element dislodge the foreign ions of the first element into deeper layers so that the desired properties of the medical device may perhaps not be realized. It is thus crucial that ions of the desired elements are implanted simultaneously and not successively.

Especially advantageous is the use of carbon ions or carbon, analogs and/or derivates as foreign ions, also oxygen ions which exhibit the capability to dislodge heavy metal ions and can be labeled as metal ion displacer in accordance with the invention.

Although foreign ions can be implanted up to a depth of about 20 μm in dependence on the substrate, significantly smaller depths of penetration may be enough to form a sufficient diffusion-inhibiting boundary layer between the surface and the interior of the substrate.

Essential for the function of the boundary layer is that the surface for ions of metals with a density greater than 4.5 g/cm³ (heavy metals) is impervious, irrespective of the position of the boundary layer. Metals with a density greater than 4.5 g/cm³ (e.g. iron, zinc, copper, manganese, tin, chromium, cadmium, lead, mercury, nickel) may, as stated above, at a certain dose promote inflammation and possibly have a toxic effect on the human organism.

While heavy metals in accordance with claim 6 may basically be present in various substrates of medical devices, this is especially the case, when the substrate is a metal. Medical special steels, nitinol but also cobalt alloys with high nickel content are possible as metals, especially for stents.

An additional measure for reducing the proliferation in the area of the device introduced into the body of the patient involves the use of therapeutic agents.

The cytostatic agent paclitaxel is known as especially effective to inhibit some cancers and to possibly effectively combat restenosis. Systematic administration of paclitaxel may trigger undesired side effects. Therefore, local application is preferred as treatment. A local treatment with paclitaxel can be more effective when administered over a prolonged period. Preferably, this period is at least as long as the normal reaction time of the body following angioplasty.

Tests have shown that a local administration of paclitaxel over a period of days or even months may be highly effective to prevent restenosis. Such a long-term time period can be replaced by a time-controlled liberation from the stent.

However, a contacting of a stent with the blood flow over an extended period may cause thrombosis that may also narrow the inner vessel diameter. A substrate surface that liberates a therapeutic agent like paclitaxel may therefore prevent restenosis but not thrombosis. It is thus desirable to provide a stent having restenosis-inhibiting properties and suitable for extended retention in the body, without experiencing the formation of a thrombus at this location.

By impregnating a stent with a restenosis-inhibiting substance, i.e. embedded underneath the surface, as opposed to a coating applied on the stent, the problems cracking, chipping and deficient adhesion of the coating and thus loss of the restenosis-inhibiting substance is eliminated. At the same time, a binding agent may be provided on the substrate to enable a bonding of anticoagulants, like, e.g., heparin, onto the binding agent and thus onto the substrate. This combination of restenosis-inhibiting substrates and anticoagulants associated to the substrate via a binding agent effectively prevents restenosis as well as thrombosis. Of course, it is possible within the scope of the invention to associate other therapeutic agents to the substrate via a binding agent, such as for example cytostatic agents like paclitaxel, if appropriate for therapeutic reasons.

Advantageously, not only the therapeutic agent is detachably associated to the binding agent. It is also possible to associate the binding agent detachably to the substrate. Thus, it is possible within the scope of the invention that the binding agent disassociates spontaneously from the substrate either after liberation of the therapeutic agent or simultaneously with the therapeutic agent. After detachment of the binding agent, e.g., following a subsiding of a body reaction in response to local injury caused, e.g. by stenting, it is primarily important to prevent allergic reactions of the body against the substrate, in particular restenosis. This function is met by the device according to the invention solely by the implanted diffusion-inhibiting boundary layer. A binding agent, like, e.g., a polymer coating, meets the ascribed task by completely releasing the therapeutic agent. The process of detachment or also dissolving is not limited to a particular time period but may be implemented also over a long time.

A preferred field for application of the medical device which is provided with a restenosis-inhibiting substance involves stents for use in coronary arteries and made of metallic material, like for example nitinol or special steel.

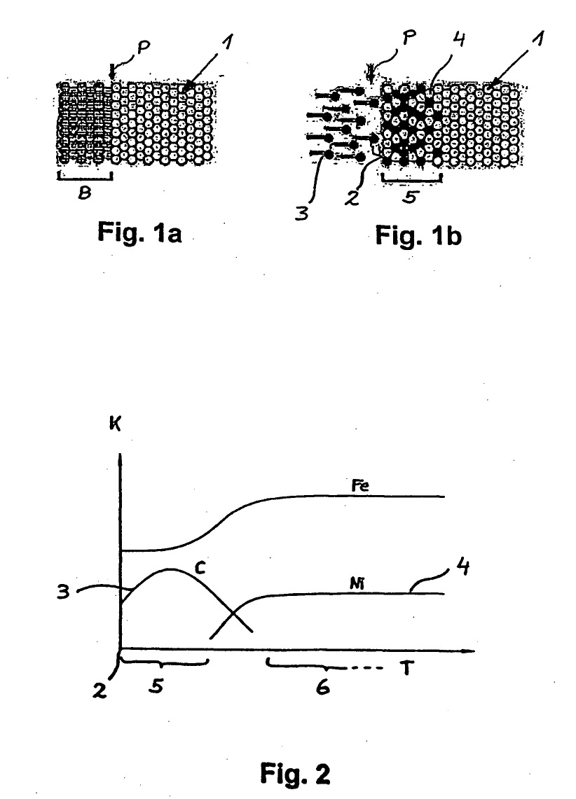

The following description explains the difference between a coating process and an ion implantation with reference to the schematic illustration of FIGS. 1a and 1b.

The surface of a coating (FIG. 1a) is additionally supplied with, for example, C-atoms. Arrow P designates hereby the original surface. As the substrate 1 and the coating B are made of two different materials with different physical properties, the adhesiveness of the coating B may pose a problem. As a consequence, a carbonized stent of special steel may be damaged during balloon dilatation in the transition zone between the coating B and the substrate 1 so that the coating B may crack or possibly even peel off.

In contrast thereto, during ion implantation (FIG. 1b), foreign ions 3 (e.g. C-ions) are directly implanted into a surface 2 of a substrate 1 (e.g. special steel). Adhesiveness is not an issue here when the substrate 1 or the stent is carbonized by the afore-described process of ion implantation. The afore-described process creates an inert surface 2 as a consequence of the firm embedment of the foreign ions 3, to prevent a diffusion of substrate ions 4, especially of heavy metal ions. This provides a protection against allergies and inflammatory reactions and contributes to the prevention of restenosis.

FIG. 2 shows the relative concentration K of implanted foreign ions 3 (C-ions) as well as the relative concentration of Fe-ions and Ni-ions in dependence on the depth T. The depth T is measured from the surface 2 of the substrate 1.

It can be recognized that the concentration K of the foreign ions 2 at a slight distance to the surface 2 initially increases up to a maximum and then progressively decreases. It is striking that the concentration of Ni-ions in the surface-proximate area approaches zero. The concentration of Ni-ions tends to approach zero even at a distance from the surface so that a diffusion barrier for Ni-ions is realized by a boundary layer 5 formed by the elevated C-ion concentration, except for a narrow transition zone between C-ions and Ni-ions. Although Ni-ions may penetrate the boundary layer up to a certain limit value of the concentration of foreign ions 3, they are, however, unable to pass through so that at least a fraction of the substrate ions, namely the Ni-ions, which are mentioned as being representative for all heavy metal ions, cannot diffuse through the boundary layer to the surface 2 of the substrate 1. The process parameters for the ion implantation are selected in any event in such a manner that a diffusion of particular substrate ions is prevented. Important hereby are a sufficient enrichment of the boundary layer with foreign ions and a certain minimum thickness of the boundary layer.

In this example, only Fe-ions and C-ions are located immediately at the surface 2 in even distribution. With increasing depth, the Fe-ion concentration rises, and following the boundary layer also the concentration of Ni-ions rises to the concentration levels existing in the interior 6 of the substrate 1.

### LIST OF REFERENCE CHARACTERS

1 substrate

2 surface of 1

3 foreign ions

4 substrate ions

5 boundary layer

6 interior of 1

B coating

K relative concentration

T depth

1. Medical device for placement in the body, in particular in a body vessel of a patient, comprising a substrate (1) suitable for long-term placement and exhibiting biocompatible properties which have been modified through bombardment of its surface (2) with foreign ions (3), characterized in that the foreign ions (3) are embedded into the substrate (1) and form a boundary layer (5) between the surface (2) and the interior (6) of the substrate to inhibit diffusion of substrate ions (4).

2. Medical device according to claim 1, characterized in that the foreign ions are a mixture of ions of several elements.

3. Medical device according to claim 1 or 2, characterized in that the foreign ions (3) are at least partially carbon ions.

4. Medical device according to one of the preceding claims 1 to 3, characterized in that the foreign ions are at least partially oxygen ions.

5. Medical device according to one of the preceding claims 1 to 4, characterized in that the foreign ions (3) are embedded in the surface (2) of the substrate (1) up to a depth of about 20 μm.

6. Medical device according to one of the preceding claims 1 to 5, characterized in that the surface (2) is impervious for ions of metals of a density greater than 4.5 g/cm³ (heavy metals).

7. Medical device according to one of the preceding claims 1 to 6, characterized in that the substrate (1) is a metal.

8. Medical device according to one of the preceding claims 1 to 7, characterized in that the surface (2) is polished.

9. Medical device according to one of the preceding claims 1 to 8, characterized in that there is provided on the surface (2) of the substrate (1) a binding agent which is detachably associated to an active medical substance.

10. Medical device according to claim 9, characterized in that the binding agent is detachably associated to the substrate (1).

11. Medical device according to claim 9, characterized in that the active medical substance is an anticoagulant.

12. Medical device according to claim 9, characterized in that the active medical substance is a cytostatic agent.
